(19) United States
(12) Patent Application Publication
(10) Pub. No.: US 2007/0212320 A1
(43) Pub. Date: Sep. 13, 2007

(54) CARE SYSTEM CONSTITUTED OF PVP AND ACRYLATE POLYMERS

(75) Inventors: Michael Demitz, Hamburg (DE); Albrecht Doerschnen, Hamburg (DE); Michaela Kohut, Hamburg (DE); Stephan Ruppert, Hamburg (DE); Harald Albrecht, Hamburg (DE); Joerg Kuether, Halstenbek (DE); Birgit Heitmann, Hamburg (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 11/573,329

(22) PCT Filed: Feb. 17, 2006
(86) PCT No.: PCT/EP06/60045

§ 371 Date: May 2, 2007

(30) Foreign Application Priority Data
Mar. 24, 2005 (DE) 10 2005 014 423.3

(51) Int. Cl. A61K 8/81

(52) U.S. Cl. 424/70.15; 424/70.16; 424/70.21

(57) ABSTRACT

The invention relates to cosmetic and dermatological detergent preparations for cleansing the skin and/or for cleaning hair and scalp. The inventive preparations comprise (a) an effective amount of one or more anionic detergents, (b) an effective amount of one or more amphoteric detergents, (c) an effective amount of one or more gelatinizing acrylate thickeners, selected from the group including the crosslinked alkali-swellable acrylate copolymers, (d) a homopolymer or copolymer based on vinylpyrrolidone, especially preferred polyvinylpyrrolidone, (e) other conventional auxiliary agents and/or additives, especially water, if desired.

The present invention relates to cosmetic cleaning compositions with a particular care performance. Cosmetic cleaning compositions are known per se. They are essentially surface-active substances or mixtures of substances which are supplied to the consumer in various preparations.

For the purposes of the present specification, care performance in the case of body-cleaning compositions is understood as meaning the achievement of a very good cleaning performance coupled with excellent mildness and product compatibility. It should be made possible for the consumer to be able to use corresponding products even a number of times daily without having to worry about adversely affecting the health of the skin.

For the purposes of the present specification, care performance in the case of hair-cleaning compositions is understood as meaning the ability of the composition to achieve, for the user thereof, clean hair, good combability of the hair, in the wet and dry state, good styleability, good feel and good hair shine.

Preparations of this type are, for example, foam and shower baths, solid and liquid soaps or so-called “syndets” (synthetic detergents), shampoos, hand-washing pastes, intimate washing compositions, special cleaning compositions for infants and babies and the like.

Surface-active substances—the most well known being the alkali metal salts of higher fatty acids, i.e. the classic “soaps”—are amphiphilic substances which can emulsify organic nonpolar substances in water.

These substances do not only wash dirt off skin and hair, they cause irritation to skin and mucosa to a greater or lesser degree depending on the choice of surfactant or the surfactant mixture.

The most common surfactant for cosmetic compositions is sodium lauryl ether sulfate. Although it has good washing power and is well tolerated by skin and mucosa, sensitive people should avoid regular contact with it.

There are indeed a large number of very mild surfactants available, although the surfactants of the prior art are either mild, but do not clean well, or else they clean well, but irritate skin or mucosa.

It was thus the aim to remedy this shortcoming.

In one particular embodiment, the present invention relates to cleaning preparations for the use as shower preparation.

Preparations of this type are also known per se. These are essentially surface-active substances or mixtures of substances which are supplied to the consumer in various preparations. Preparations of this type are generally characterized by a greater or lesser water content, but can also be, for example, in the form of a concentrate.

In general, preparations which are intended for the shower bath do not differ or barely differ from bathtub preparations apart from the fact that in the case of shower preparations products of higher viscosity are preferred which, following removal from the container, do not run out of the hand. This is of less practical importance in the case of bathtub preparations.

Even a simple bath of water without the addition of surfactants results firstly in swelling of the horny layer of the skin, this degree of swelling depending, for example, on the bath time and temperature. At the same time, water-soluble substances, e.g. water-soluble dirt constituents, but also endogenous substances which are responsible for the water-binding capacity of the horny layer are washed off or out. In addition, as a result of endogenous surface-active substances, fats in the skin are also dissolved and washed out to a certain extent. Following the initial swelling, this causes subsequent considerable drying out of the skin, which can be further intensified by washing-active additives.

In the case of healthy skin, these processes are generally harmless since the protective mechanisms of the skin can compensate directly for such slight disturbances to the upper skin layers. However, even in the case of nonpathological deviations from the normal status, e.g. as a result of environment-induced wear damage and/or irritations, photo-damage, aging skin, etc., the protective mechanism of the skin surface is disrupted. Under certain circumstances, it is no longer able, by itself, to perform its function and has to be regenerated through external measures.

It was thus an object of the present invention to remedy this shortcoming of the prior art. In addition, it was an object of the invention to provide bathtub and shower bath preparations which, on the one hand, have a high care effect without, on the other hand, the cleaning effect taking second place to it.

The present invention also relates to washing-active hair cosmetic preparations, commonly referred to as shampoos. In particular, the present invention relates to hair cosmetic active ingredient combinations and preparations for the care of the hair and the scalp.

Even washing the hair with aggressive surfactants can stress the hair, at least diminish its appearance or the appearance of the hairstyle overall. For example, certain water-soluble hair constituents (e.g. urea, uric acid, xanthine, keratin, glycogen, citric acid, lactic acid) can be leached out by washing the hair.

However, the prior art lacked shampoo formulations which give care to the damaged hair in a satisfactory way. The object was therefore to also overcome these disadvantages of the prior art.

Customary cosmetic and dermatological preparation forms which are becoming evermore widespread, especially recently, are gels.

Cosmetic gels enjoy immense popularity amongst consumers. Since most of them are transparent, often colored or may just as often be colorless and clear, they offer the cosmetics developer additional design opportunities, some of which are functional in character, whilst some merely serve to improve the external appearance. Thus, for example, it is possible to impart interesting optical effects to the product, which is then usually offered to the observer in transparent packaging, through incorporated color pigments, gas bubbles and the like, or alternatively, larger objects.

It is desirable that these objects remain stationary in the gel formulation and do not sink to the bottom or migrate undesirably in any other way in the formulations, especially if it is desired that the incorporated object or objects—be they discernible as such with the naked eye, or be they present in microscopic dimensions, but arranged interestingly—for example in the form of artificially produced colored swirls—then produce visible shapes.

Liquids can differ with regard to their rheological properties as a result of their flow and deformation behavior. As a result of external sources, ideally elastic bodies undergo elastic deformation which spontaneously and completely reverses when the external force is removed. The shape of ideally viscous bodies is changed irreversibly as a result of external forces. The increasing deformation is termed flow. Most liquids are neither ideally viscous nor ideally elastic, but exhibit both viscous and elastic properties and are thus termed viscoelastic substances.

In the large majority of viscoelastic solutions, dispersed particles or gel bubbles will always sediment or rise, respectively. They have an ultimate structural relaxation time. This means that the networks in these systems react to a deformation with a corresponding shear stress. However, this will relax to zero in a finite time so that the whole solution reverts to a stable steady state without strain. This also means that these solutions have a defined zero-shear viscosity and thus reach a constant viscosity at low shear rates.

In contrast to these systems, however, there are also those in which dispersed particles or gas bubbles do not sediment. It is noticeable that these systems only flow above a characteristic value. This value is called yield point. Closer inspection of the rheological properties of these systems indicates that the storage modulus is independent of the oscillation frequency over the whole frequency range and is always significantly greater than the loss modulus.

By contrast, the complex viscosity does not reach a constant value even at the lowest frequencies, but continues to increase.

Carbopol gels are crosslinked acrylic acid polymers having a large number of carboxyl groups. In dissolved form, these structures bind water. Neutralization of the carboxyl groups leads, as a result of their electrostatic repulsion, to expansion and thus swelling of the polymer chains. In this state, the carbopol gels achieve their typical rheological properties, such as, for example, the formation of a yield point.

The effect of forming a yield point is thus based on the electrostatic repulsion of the carboxyl groups. Additional electrolytes shield these charges. As a result, the networks collapse, the yield point breaks down and particles or gas bubbles can no longer be held in suspension.

Surfactants act as electrolytes. It has thus to date not been possible to formulate high-foam cleaning products with a correspondingly high content of surfactant which comprised, as base, clear carbopol gels having a yield point.

Although the prior art recognizes corresponding systems containing xanthan gum (e.g. EP-A 738 509), they have poorer cosmetic properties in terms of the feel on the skin during and following application. Moreover, it is only possible to obtain lower viscosities for the same use concentration. The formulation of a gel which has suitable flow properties does not usually present the person skilled in the art with any major problems, except where high surfactant concentrations are to be achieved—usually a basic requirement for cleaning products. The disadvantage of such high surfactant concentrations is that in most cases only hazy, cloudy or even opaque products are obtained.

WO 01/19946 discloses washing-active formulations which comprise a conditioner besides a gel former. WO 01/176552 discloses washing-active formulations which relates to a combination of certain thickeners with acyl glutamates. However, these specifications were unable to point the way to the present invention.

The consistency of the product influences the amount of foam and the foam creaminess, particularly at the point of foam development and thus the acceptance of a product by the consumer. Even while spreading the product and during foam development in the hand or in the hair, the consumer evaluates the product with regard to the care performance to be expected. A high consistency and a creamy, rich foam development is synonymous here with the expectation of the extensive care performance.

In order to achieve increased consistency in a customary shampoo formulation, shower gel formulation or face-cleansing formulation, the customary thickeners can be used. However, these do not lead to an acceptable cosmetic flow and removal behavior. It is more advantageous here to use special thickening systems based on polyacrylates. Using these systems, it is possible to construct even surfactant-containing formulations (shampoos) with yield points, meaning that small, visible particles can be kept in suspension in the products. However, these systems do not lead to shampoos with an acceptable care performance or shower gels and face cleansers with adequate skincare performance or sensory properties. Even by adding customary conditioning substances, such as cationic polymers (also called polymeric quats), the skin and hair care performance, in particular the combability in the wet and dry hair, can not be adjusted to a shampoo desired for dry/stressed hair.

It was therefore a further object of the present invention to find ways which enable polyacrylates-containing shampoos containing cationic polymers with simultaneous comparatively good care performance to be produced.

It was thus also the aim to remedy this disadvantage of the prior art.

EP 0636357 discloses hair-treatment compositions which comprise combinations of amphoteric surfactants, cationic surfactants and PVP. In the preparations according to the invention, it is possible to dispense with the use of cationic surfactants in order to achieve the desired care performance.

EP 200620 discloses hair-treatment compositions comprising ethoxylated triglycerides, PVP and phenyltrimethicone. In the preparations according to the invention, the use of phenyltrimethicone can be dispensed with in order to achieve the desired care performance.

DE 3336760 discloses cleaning preparations comprising alkyl polyglucosides and PVP. In the preparations according to the invention, the use of alkyl polyglucosides can be dispensed with in order to achieve the desired care performance.

U.S. Pat. No. 4,676,978 discloses hair-cleaning compositions comprising lauryl ether sulfate, guar quat, polyethylene glycol polyamine and PVP. In the preparations according to the invention, the use of polyethylene glycol polyamine can be dispensed with in order to achieve the desired care performance.

WO 02058646 discloses hair-treatment compositions comprising a protective active ingredient, conditioners and copolymers with PVP units. In the preparations according to the invention, the use of these special copolymers can be dispensed with in order to achieve the desired care performance.

WO 02/65996 discloses preparations with two different anionic associative polymers but which do not achieve the care performance according to the invention on hair and/or skin.

US 2004/42448 discloses a system for thickening and rheological adjustment with a complicated terpolymer and a polysaccharide, although this does not achieve the care performance according to the invention on hair and/or skin.

DE 3414090 discloses preparations with an anionic polymer and special surface-active agents, although these do not achieve the care performance according to the invention on hair and/or skin.

EP 1090631 discloses preparations containing a thickener and a transparency agent (polyols). In the preparations according to the invention, the use of polyols can be dispensed with.

EP 965322 discloses surfactant-containing preparations with certain polymers in combination with ethoxylated alcohols, although these have nothing in common with the polymer combinations according to the invention. In the preparations according to the invention, the use of ethoxylated alcohols can be dispensed with.

EP 1010422 discloses surfactant-containing preparations with two different thickener systems which are constructed from polyacrylates and polyalkoxy esters. In the preparations according to the invention, the use of polyalkoxy esters can be dispensed with.

EP 1291001 discloses preparations with certain acrylate copolymers and oils in concentrations greater than 0.2%. In the preparations according to the invention, the use of these oils in concentrations greater than or equal to 0.2% can be dispensed with in order to obtain the desired care performance.

Surprisingly, it has been found, and herein lies the attainment of these objects, that cosmetic and dermatological washing-active preparations for cleaning the skin and/or for hair and scalp cleaning, comprising

(a) an effective amount of one or more anionic surfactants,

(b) an effective amount of one or more amphoteric surfactants,

(c) an effective amount of one or more gel-forming acrylate thickeners chosen from the group of crosslinked alkali-swellable acrylate copolymers,

(d) a homopolymer or copolymer based on vinylpyrrolidone, particularly preferably polyvinylpyrrolidone,

(e) if desired further customary auxiliaries and/or additives, in particular water, overcome the disadvantages of the prior art.

Here, it is advantageous if the formulations additionally comprise cationic polymers.

Here, it is advantageous if the anionic surfactant chosen is lauryl ether sulfate and/or myreth ether sulfates and the amphoteric surfactant chosen is cocoamidopropylbetaine.

Here, it is advantageous if the total amount of one or more surfactants used according to the invention is chosen from the range from 0.1-25% by weight, preferably 1-12% by weight, particularly preferably 2-10% by weight, in each case based on the total weight of the preparations.

Here, it is advantageous if the total amount of one or more anionic surfactants used according to the invention is chosen from the range from 5 to 15% by weight, preferably 6 to 12% by weight, in each case based on the total weight of the preparations.

Here, it is advantageous if the total amount of one or more amphoteric surfactants used according to the invention is chosen from the range from 0.5 to 10% by weight, preferably 1 to 5% by weight, in each case based on the total weight of the preparations.

Here, it is advantageous if the acrylate thickener used is a copolymer consisting of a) an acrylate monomer chosen from acrylic acid, methacrylic acid, itaconic acid, fumaric acid, crotonic acid, aconitic acid or maleic acid, b) an a,b-ethylenically unsaturated monomer of the general formula CH₂═CXY where X═H, CH₃, —C1-C30-alkyl, —CH₂—C(═O)O(CH₂—CH₂—O)ₓ—R³, —CH₂—C(═O)NH(CH₂—CH₂—O)ₓ—R³, —CH₂—CH₂═(CH₂—CH₂—O)ₓ—R³ where x=1-100 and R³=C1-C30 alkyl or Cl and Y=—COOR, —C₆H₄R, —CN, —CONH₂, —Cl, —NC₄H₆O, —NH(CH₂)₃COOH, —NHCOCH₃, —CONHC(CH₃)₃, —CON(CH₃)₂, —CH═CH₂, C1-C18-alkyl, hydroxy-C1-C18-alkyl, —C(═O)O(CH₂—CH₂—O)ₓ—R³, —C(═O)NH(CH₂—CH₂)ₓ—R³, —CH2=(CH₂—CH₂—O)ₓ—R³ where x=1-100 and R³=C1-C30-alkyl or of the formula CH₂═CH(OCOR²) where R²=C1-C18 alkyl or of the formula CH₂═CH₂ or CH₂═CHCH₃ and c) a polyunsaturated component which is suitable for partial crosslinking.

Here, it is advantageous if the total amount of one or more acrylate thickeners is chosen from the range 0.1-8.0% by weight, preferably 0.3-6% by weight, particularly preferably 0.5-4% by weight, extraordinarily preferably 1.5-3% by weight, based on the total weight of the preparations.

Here, it is advantageous if the polyvinylpyrrolidone is present in contents of from 0.01 to 3% by weight, particularly preferably 0.1 to 1% by weight, extraordinarily preferably 0.1 to 0.9% by weight.

Here, it is advantageous if the cationic polymer used is polyquaternium-7, polyquaternium-10, polyquaternium-44 or guar quats or a combination of polyquats and guar quat.

Here, it is advantageous if the cationic polymer is used in concentrations of from 0.01 to 0.6% by weight, particularly preferably 0.1 to 0.3% by weight.

Here, it is advantageous if the cationic polymer used is polyquaternium-10 and/or guar quat.

Here, it is advantageous if the cationic polymer used is exclusively polyquaternium-1 0.

Here, it is advantageous if at least one nonionic surfactant is present which has an HLB of from 10 to 20.

The acrylate thickener to be used advantageously according to the invention is advantageously a product which is sold by Noveon under the name Aqua SF-1 (INCI: Acrylates Copolymer). It is a slightly crosslinked acrylate copolymer swellable by alkalis which comprises three structural components, namely one or more carboxylic acid monomers having 3 to 10 carbon atoms, one or more vinyl monomers, and, as third component, mono- or polyunsaturated monomers.

Also advantageous are compounds which the INCI name “Acrylates/Ceteth-20 Itaconate Copolymer” (available under the trade names Structure 2001® from National Starch), which the INCI name “Acrylates Copolymer” (available under the trade names SALCARE SC81® from Ciba), which the INCI name “Acrylates Copolymer” (available under the trade names POLYGEL W400® from 3V), which the INCI name “Acrylates Copolymer” (available under the trade names VISCOLAM MAC-7® from Lamberti) and which the INCI name “Acrylates/Beheneth-25 Methacrylate Copolymer” (available under the trade names TINOVIS GTC®) from Ciba) and similar polymers.

For the purposes of the invention, anionic surfactants generally have, as functional groups, carboxylate, sulfate or sulfonate groups. In aqueous solution, they form negatively charged organic ions in an acidic or neutral medium. Cationic surfactants are characterized almost exclusively by the presence of a quaternary ammonium group. In aqueous solution, they form positively charged organic ions in an acidic or neutral medium. Amphoteric surfactants contain both anionic and cationic groups and accordingly behave in aqueous solution as anionic or cationic surfactants depending on the pH. In a strongly acidic medium, they have a positive charge, and in an alkali medium they have a negative charge. By contrast, in a neutral pH range, they are zwitterionic, as the following example is intended to illustrate:

| | | |
|---|---|---|
| RNH₂⁺CH₂CH₂COOH X⁻ | (at pH = 2) | X⁻ = any anion, e.g. Cl⁻ |
| RNH₂⁺CH₂CH₂COO⁻ | (at pH = 7) | |
| RNHCH₂CH₂COO⁻ B⁺ | (at pH = 12) | B⁺ = any cation, e.g. Na⁺ |

Typical nonionic surfactants are polyether chains. Nonionic surfactants do not form ions in an aqueous medium.

A. Anionic Surfactants

Anionic surfactants to be used advantageously according to the invention are acylamino acids (and salts thereof), such as

1. acyl glutamates, for example sodium acyl glutamate, di-TEA palmitoyl aspartate and sodium caprylic/capric glutamate,

2. acyl peptides, for example palmitoyl-hydrolyzed milk protein, sodium cocoyl-hydrolyzed soya protein and sodium/potassium cocoyl-hydrolyzed collagen,

3. sarcosinates, for example myristoyl sarcosine, TEA lauroyl sarcosinate, sodium lauroyl sarcosinate and sodium cocoyl sarcosinate,

4. taurates, for example sodium lauroyl taurate and sodium methylcocoyl taurate,

5. acyl lactylates, lauroyl lactylate, caproyl lactylate

6. alaninates

Carboxylic acids and derivatives, such as

1. carboxylic acids, for example lauric acid, aluminum stearate, magnesium alkanolate and zinc undecylenate,

2. eEster carboxylic acids, for example calcium stearoyl lactylate, laureth-6 citrate and sodium PEG-4 lauramide carboxylate,

3. ether carboxylic acids, for example sodium laureth-13 carboxylate and sodium PEG-6 cocamide carboxylate,

phosphoric acid esters and salts, such as, for example, DEA oleth-10 phosphate and dilaureth-4 phosphate,

Sulfonic acids and salts, such as

1. acyl isethionates, e.g. sodium/ammonium cocoyl isethionate,

2. alkylarylsulfonates,

3. alkylsulfonates, for example sodium cocomonoglyceride sulfate, sodium C₁₂₋₁₄ olefinsulfonate, sodium lauryl sulfoacetate and magnesium PEG-3 cocamide sulfate,

4. sulfosuccinates, for example dioctyl sodium sulfosuccinate, disodium laureth sulfosuccinate, disodium lauryl sulfosuccinate and disodium undecylenamido-MEA sulfosuccinate

and

sulfuric acid esters, such as

1. alkyl ether sulfate, for example sodium, ammonium, magnesium, MIPA, TIPA laureth sulfate, sodium myreth sulfate and sodium C₁₂₋₁₃-pareth sulfate,

2. alkyl sulfates, for example sodium, ammonium and TEA lauryl sulfate.

B. Amphoteric Surfactants

Amphoteric surfactants to be used advantageously according to the invention are

1. acyl-/dialkylethylenediamine, for example sodium acyl amphoacetate, disodium acyl amphodipropionate, disodium alkyl amphodiacetate, sodium acyl ampho-hydroxypropylsulfonate, disodium acyl amphodiacetate and sodium acyl ampho-propionate,

2. N-alkylamino acids, for example aminopropylalkylglutamide, alkylaminopropionic acid, sodium alkylimidodipropionate and lauroamphocarboxyglycinate.

Advantageous nonionic surfactants with an HLB value of from 10 to 20 may, for example, be:

PEG-20 almond oil glyceride, PEG-20 evening primrose oil glyceride, PEG-60 hydrogenated castor oil, polysorbate 60, PEG-20 sorbitan isostearate, PEG-40 hydrogenated castor oil, polysorbate 80, sucrose cocoate, PEG-45 palm kernel oil glycerides, PEG-45 thistle oil glyceride, PEG-60 evening primrose oil glyceride, PEG-42 babassu oil glyceride, PEG-20 ricinus oleate, steareth-21, oleth-20, polysorbate 40, laureth-9, laureth-15, ceteareth-20, ceteth-20, PEG-12 laurate, PEG-24 stearate, PEG-20 stearate, PEG-20 oleate, PEG-75 lanolin, laneth-40, PEG-8 dilaurate, polysorbate 65, PEG-7 glyceryl cocoate, laneth-10, PEG-12 oleate, polyglyceryl-6 laurate, polyglyceryl-10 laurate, polyglyceryl-10 myristate, polyglyceryl-10 stearate, polyglyceryl-10 oleate, polyglyceryl-10 isostearate, polyglyceryl-10 diisostearate, PEG-15 glyceryl oleate, PEG-60 sorbitan tetraoleate, PEG-10 oleyl ether.

Film formers advantageous according to the invention may be chosen here from the compounds listed in the table.

TABLE 1
Film formers advantageous according to the invention

| | | | Example (trade |
|---|---|---|---|
| INCI name | CAS number | Polymer type | name) |
| Polyquaternium-2 | CAS 63451-27-4 | Urea, N,N'-bis[3-(di- | Mirapol ® A-15 |
| | | methylamino)propyl]polymer | |
| | | with 1,1'-oxybis(2- | |
| | | chloroethane) | |
| Polyquaternium-5 | CAS 26006-22-4 | Acrylamide, β-methacryl- | |
| | | oxyethyltriethylammonium | |
| | | methosulfate | |
| Polyquaternium-6 | CAS 26062-79-3 | N,N-Dimethyl-N-2-propenyl- | Merquat ® 100 |
| | | 2-propenaminium chloride | |
| Polyquaternium-7 | CAS 26590-05-6 | N,N-Dimethyl-N-2-propenyl- | Merquat ® S |
| | | 2-propenaminium chloride, | |
| | | 2-propenamide | |
| Polyquaternium-10 | CAS 534568-66-4, | Quaternary ammonium salt | Celquat ® SC- |
| | 55353-19-0, 54351- | of hydroxyethylcellulose | 230M |
| | 50-7, 68610-92-4, | | |
| | 81859-24-7 | | |
| Polyquaternium-11 | CAS 53633-54-8 | Vinylpyrrolidone/dimethyl- | Gafquat ® 755N |
| | | aminoethyl methacrylate | |
| | | copolymer/diethyl sulfate | |
| | | reaction product | |
| Polyquaternium-16 | CAS 29297-55-0 | Vinylpyrrolidone/vinyl- | Luviquat ® |
| | | imidazolinium ethochloride | HM552 |
| | | copolymer | |
| Polyquaternium-17 | CAS 90624-75-2 | | Mirapol ® AD-1 |
| Polyquaternium-19 | CAS 110736-85-1 | Quaternized water-soluble | |
| | | polyvinyl alcohol | |
| Polyquaternium-20 | CAS 110736-86-2 | Water-dispersible | |
| | | quaternized polyvinyl | |
| | | octadecyl ether | |
| Polyquaternium-21 | | Polysiloxane-polydimethyl- | Abil ® B 9905 |
| | | dimethylammonium acetate | |
| | | copolymer | |
| Polyquaternium-22 | CAS 53694-17-0 | Dimethyldiallylammonium | Merquat ® 280 |
| | | chloride/acrylic acid | |
| | | copolymer | |
| Polyquaternium-24 | CAS 107987-23-5 | Polymeric quaternary | Quartisoft ® |
| | | ammonium salt of hydroxy- | LM-200 |
| | | ethylcellulose | |
| Polyquaternium-28 | CAS 131954-48-8 | Vinylpyrrolidone/meth- | Gafquat ® |
| | | acrylamidopropyltrimethyl- | HS-100 |
| | | ammonium chloride | |
| | | copolymer | |
| Polyquaternium-29 | CAS 92091-36-6, | Chitosan which has been | Lexquat ® CH |
| | 148880-30-2 | reacted with propylene oxide | |
| | | and quaternized with | |
| | | epichlorohydrin | |
| Polyquaternium-31 | CAS 136505-02-7, | Polymeric, quaternary | Hypan ® QT 100 |
| | 139767-67-7 | ammonium salt which is | |
| | | prepared by reacting DMAPA | |
| | | acrylate/acrylic | |
| | | acid/acrylonitrogen | |
| | | copolymer and diethyl sulfate | |
| Polyquaternium-32 | CAS 35429-19-7 | N, N, N-Trimethyl-2-{[82- | |
| | | methyl-1-oxo-2-propenyl)- | |
| | | oxy]ethanaminium chloride, | |
| | | polymer with 2-propenamide | |
| Polyquaternium-37 | CAS 26161-33-1 | | |
| Polyquaternium-44 | | Copolymeric quaternary | |
| | | ammonium salt of vinyl- | |
| | | pyrrolidone and quaternized | |
| | | imidazoline | |

Further film formers advantageous according to the invention are cellulose derivatives and quaternized guar gum derivatives, in particular guar hydroxypropylammonium chloride (e.g. Jaguar Excel®, Jaguar C 162® from Rhodia, CAS 65497-29-2, CAS 39421-75-5).

Nonionic poly-N-vinylpyrrolidone/polyvinyl acetate copolymers (e.g. Luviskol VA 64W®, BASF), anionic acrylate copolymers (e.g. Luviflex soft®, BASF), and/or amphoteric amide/acrylate/methacrylate copolymers (e.g. Amphomer®, National Starch) can also be used advantageously according to the invention as film formers.

Film formers preferred according to the invention are polyquaternium-10, polyquaternium-22 and polyquaternium-44 and Jaguar Excel®. According to the invention, particular preference is given to polyquaternium-10 (e.g. Ucare Polymer JR-125® and Ucare Polymer JR-400), Amerchol) in combination with Jaguar Excel®.

The washing-active preparations according to the invention are generally characterized by a water content of 95-5% by weight, based on the total weight of the preparations, and are gels.

Advantageously, the preparations—if they are gel-like preparations with a yield point—are formulated so that they have a yield point of 0.5-20 Pa, preferably 1-6 Pa.

The yield point is regarded as the critical shear stress of the flow curve. It can be ascertained according to the invention as follows:

The flow curve is measured on a shear-stress-controlled rheometer at 25° C.±1° C. with 25 mm plate/plate geometry at a gap between 0.8 mm and 1.2 mm, with charging being carried out in a structure-preserving manner. A suitable constant shear stress gradient is pregiven and, before the test, a corresponding structure recovery time is observed and the critical shear stress at the maximum of the flow curve is stated.

Advantageously, the preparations are formulated so that they have a tan δ of 0.05-0.6, preferably 0.1-0.5.

According to the invention, tan δ is understood as meaning the quotient of the loss modulus and the storage modulus. The tan δ is ascertained as follows:

Loss and storage moduli are measured by a dynamic frequency test on a shear-stress-controlled rheometer at 40° C.±1° C. with 25 mm plate/plate geometry at a gap between 0.8 mm and 1.2 mm, charging being carried out in a structure-preserving manner. The frequency test is carried out according to the prior art with an appropriate structure recovery time before the test, and the tan δ in the frequency range between 0.05 rad/s and 3.0 rad/s is quoted, preferably between 0.08 rad/s and 1.0 rad/s.

The yield point can be raised by increasing the gel former concentration. For the purposes of the invention, the use of nonionic film formers has proven advantageous, in particular of polyvinylpyrrolidone (PVP), very particularly of PVP which is supplied under the trade name Luviskol K 30 powder (BASF). Preferred use concentrations are 0.01-3% by weight, particularly preferably 0.1-1% by weight.

Pearlescent systems which may be used are in principle all customary known pearlescent raw materials. The use of PEG-3 distearate is particularly advantageous. It is very particularly advantageous if this pearlescent raw material has a particle size such that 90% of the volume fractions have a particle size of less than 50 μm. The use concentrations of the pearlizer are advantageously 5-15% by weight.

Instead of the pearlescent system, an opacifier can also be used. The use of mixtures consisting of glycol distearate, cocoglucoside, glyceryl oleate, glyceryl stearates, as are supplied under the trade name Lamesoft TM Benz (Cognis) is particularly advantageous. A combination of pearlizer and opacifier can also be used advantageously.

In the case of face cleaning—i.e. not in the case of hair and scalp cleaning—combinations of myreth sulfate with cocamidopropylbetaine are to be used particularly advantageously. Myreth sulfate is to be used particularly advantageously in concentrations of 1-10% by weight and cocamidopropylbetaine in concentration of 0.5-10% by weight. It is very particularly advantageous to use myreth sulfate in concentrations of 2-6% by weight and cocamidopropylbetaine in concentrations of 1-3% by weight. In addition, 0.5-5% alkyl polyglucosides can be used as an important cosurfactant.

The cosmetic and dermatological preparations according to the invention can comprise cosmetic auxiliaries, as are usually used in such preparations, e.g. preservatives, bactericides, perfumes, substances for preventing foaming, dyes, pigments which have a coloring effect, thickeners, moisturizing and/or humectant substances, fats, oils, waxes or other customary constituents of a cosmetic or dermatological formulation, such as alcohols, polyols, polymers, foam stabilizers, electrolytes, organic solvents or silicone derivatives.

Preparations according to the invention are advantageously buffered to a pH range >4.2, particularly preferably >4.5, particularly preferably 4.8-7.5.

The cosmetic and/or dermatological preparations according to the invention are prepared in the manner customary to the person skilled in the art, in most cases by suspending the interface-active substances used according to the invention with uniform stirring and, if necessary, with heating and, if desired, homogenizing them, optionally combining them with further lipid components and optionally with one or more further emulsifiers, then mixing the oil phase with the aqueous phase into which a thickener has optionally been incorporated and which preferably has approximately the same temperature as the oil phase, if desired, homogenizing them and allowing them to cool to room temperature. After cooling to room temperature, repeated homogenization can take place, particularly if volatile constituents are still to be incorporated.

The examples below are intended to illustrate the present invention without limiting it. Unless stated otherwise, all of the quantitative data, fractions and percentages are based on the weight and the total amount or on the total weight of the preparations.

### EXAMPLES

Aqua SF-1—if used—is diluted with part of the water phase and added to the surfactant phase with stirring. The other formulation constituents are then added with stirring, and the pH is adjusted using NaOH. During all of this, stirring is carried out with the slightest possible shearing.

Shampoos

| | 1 | 2 | 3 | 4 | 5 | 6 |
|---|---|---|---|---|---|---|
| Sodium lauryl ether sulfate | 4 | 4 | 5 | 4 | 6 | 8 |
| Cocamidopropylbetaine | 4 | 3.5 | 3.5 | 3.5 | 4 | 4 |
| Polyquaternium-10 | 0.1 | 0.3 | 0.2 | 0.3 | 0.1 | — |
| Guar hydroxypropyltrimonium | — | — | — | — | — | 0.1 |
| chloride | | | | | | |
| Acrylates copolymer (Aqua SF-1) | 3.0 | 1.5 | 2.0 | 2.5 | 2.5 | 2.4 |
| Polyvinylpyrrolidone | 0.5 | 0.5 | 0.4 | 0.5 | 0.6 | 0.5 |
| PEG-40 hydrogenated castor oil | 0.2 | 0.6 | 0.2 | 0.8 | 0.6 | 1.2 |
| Pearlescence (PEG-3 distearate) | 1.5 | 1.5 | — | — | — | — |
| Opacifier Lamesoft TM Benz | — | 4.0 | 3.0 | 4.0 | 5.0 | 4.0 |
| (glycol distearate, cocoglucoside, | | | | | | |
| glyceryl oleate, glyceryl stearate) | | | | | | |
| Methylparaben | 0.4 | 0.4 | 0.4 | 0.4 | 0.4 | 0.4 |
| Propylparaben | 0.2 | 0.1 | 0.2 | 0.2 | 0.2 | 0.2 |
| Phenoxyethanol | 0.5 | 0.6 | 0.6 | 0.4 | 0.6 | 0.6 |
| EDTA | 0.3 | 0.2 | 0.2 | 0.1 | 0.2 | 0.2 |
| Sodium hydroxide solution | q.s. | q.s. | q.s. | q.s. | q.s. | q.s. |
| Citric acid | q.s. | q.s. | q.s. | q.s. | q.s. | q.s. |
| Perfume | q.s. | q.s. | q.s. | q.s. | q.s. | q.s. |
| Water | ad 100 | ad 100 | ad 100 | ad 100 | ad 100 | ad 100 |

Aqua SF-1 is diluted with some of the water phase and added to the surfactant phase with stirring. The other formulation constituent apart from NaOH and the suspension bodies are then added with stirring. After the pH has been adjusted, the suspension bodies are stirred into the finished gel base with the slightest possible shearing.

Preparations for Cleaning the Body:

| | 1 | 2 | 3 | 4 | 5 | 6 |
|---|---|---|---|---|---|---|
| Sodium laureth sulfate | 11.0% | 9.5% | 12.0% | 9.0% | 12% | 6% |
| Cocamidopropylbetaine | 4.0% | 4.5% | 1.1% | | | 5% |
| Sodium cocoamphoacetate | | | | 4.5% | 3.5% | |
| Sodium cocoyl glutamate | 1.5% | 2.5% | 0.8% | 0.5% | 1.0% | |
| Acrylates copolymer (Aqua SF-1) | 3.0% | 2.5% | 2.2% | | | |
| Acrylates/C10-30 alkyl acrylate | | | | | | |
| crosspolymer | | | | | | |
| Magnesium aluminum silicate | | | | 3.0% | 2.3% | |
| Polyvinylpyrrolidone | 0.3% | 0.9% | 0.1% | 0.3% | 0.6% | 0.4% |
| Hydroxypropyl guarhydroxy- | | | | 0.1% | 0.1% | |
| propyltrimonium chloride | | | | | | |
| PEG-6 caprylic/capric glycerides | | | 1.0% | | | |
| PEG-40 hydrogenated castor oil | 0.5% | 0.5% | 0.5% | 0.5% | 0.5% | 1.0% |
| Glycol distearate | 0.3% | | | | | |
| Glycerol | 0.2% | | | | | 1.5% |
| PEG-7 glyceryl cocoate | | 1.5% | | | | 1.0% |
| Styrene/acrylate copolymer | | | 1.0% | | | 2.5% |
| PEG-3 distearate | | | | 2.0% | 2.0% | |
| Trisodium EDTA | | 0.2% | | | 0.2% | |
| Benzophenone-4 | | 0.1% | | | 0.1% | |
| Polyethylene | 1.5% | | | 5% | 2% | |
| Pigments | q.s. | q.s. | q.s. | q.s. | q.s. | |
| Polyquaternium-10 | | 0.2% | | | | 0.1% |
| Polyquaternium-7 | | | 0.4% | 0.2% | | 0.2% |
| Preservatives | q.s. | q.s. | q.s. | q.s. | q.s. | q.s. |
| Dye | q.s. | q.s. | q.s. | q.s. | q.s. | q.s. |
| Citric acid | q.s. | q.s. | q.s. | q.s. | q.s. | q.s. |
| NaOH | q.s. | q.s. | q.s. | q.s. | q.s. | q.s. |
| Perfume | q.s. | q.s. | q.s. | q.s. | q.s. | q.s. |
| Water | ad 100 | ad 100 | ad 100 | ad 100 | ad 100 | ad 100 |

Preparations for Cleaning the Face:

| | 1 | 2 | 3 | 4 | 5 |
|---|---|---|---|---|---|
| Sodium myreth sulfate | 2% | — | 3% | 5% | 2% |
| Sodium laureth sulfate | — | 2% | 1% | — | 2% |
| Cocamidopropylbetaine | 1% | 2% | 1% | 1% | 1% |
| Acrylates copolymer | 0.3% | 0.5% | 0.2% | 0.2% | 1% |
| (Aqua SF-1) | | | | | |
| Polyvinylpyrrolidone | 0.2% | 0.5% | 0.3% | 0.2% | 0.1% |
| Sodium hydroxide | 0.5% | 0.5% | 0.5% | 0.5% | 0.5% |
| PEG-7 glyceryl cocoate | 1% | 0.5% | — | — | — |
| Na3HEDTA | 0.5% | 0.5% | 0.5% | 0.5% | 0.5% |
| Polyquaternium-10 | 0.1% | — | 0.2% | — | — |
| Licochalcone A | 0.025% | — | 0.05% | — | 0.02% |
| Ubiquinone | — | 0.01% | — | — | 0.025% |
| Polyethylene | 1% | 0.5% | 0.1% | — | — |
| PEG-40 hydrogenated | — | 0.5% | — | 1% | — |
| castor oil | | | | | |
| Phenoxyethanol | 0.5% | 0.5% | 0.5% | 0.5% | 0.5% |
| Parabens | 0.2% | 0.2% | 0.2% | 0.2% | 0.2% |
| Perfume | q.s. | q.s. | q.s. | q.s. | q.s. |
| Water | ad 100 | ad 100 | ad 100 | ad 100 | ad 100 |

1.-14. (canceled)

15. A cosmetic or dermatological washing-active preparation for cleansing skin and/or hair and scalp, wherein the preparation comprises
(a) an effective amount of one or more anionic surfactants;
(b) an effective amount of one or more amphoteric surfactants;
(c) an effective amount of one or more gel-forming acrylate thickeners selected from crosslinked alkali-swellable acrylate copolymers; and
(d) a homo- or copolymer of vinylpyrrolidone.

16. The preparation of claim 15, wherein the preparation further comprises water.

17. The preparation of claim 15, wherein (d) comprises polyvinylpyrrolidone.

18. The preparation of claim 15, wherein (a) comprises at least one of lauryl ether sulfate and myreth ether sulfate.

19. The preparation of claim 15, wherein (b) comprises cocoamidopropylbetaine.

20. The preparation of claim 15, wherein the preparation further comprises one or more cationic surfactants.

21. The preparation of claim 15, wherein the preparation comprises a total of from 0.1% to 25% by weight of surfactants, based on a total weight of the preparation.

22. The preparation of claim 21, wherein the preparation comprises a total of from 1% to 12% by weight of surfactants.

23. The preparation of claim 21, wherein the preparation comprises a total of from 2% to 10% by weight of surfactants.

24. The preparation of claim 15, wherein the preparation comprises a total of from 5% to 15% by weight of the one or more anionic surfactants, based on a total weight of the preparation.

25. The preparation of claim 24, wherein the preparation comprises a total of from 6% to 12% by weight of the one or more anionic surfactants.

26. The preparation of claim 15, wherein the preparation comprises a total of from 0.5% to 10% by weight of the one or more amphoteric surfactants, based on a total weight of the preparation.

27. The preparation of claim 26, wherein the preparation comprises a total of from 1% to 5% by weight of the one or more amphoteric surfactants.

28. The preparation of claim 15, wherein the preparation comprises a total of from 0.1% to 8.0% by weight of the one or more acrylate thickeners, based on a total weight of the preparation.

29. The preparation of claim 28, wherein the preparation comprises a total of from 0.3% to 6% by weight of the one or more acrylate thickeners.

30. The preparation of claim 28, wherein the preparation comprises a total of from 0.5% to 4% by weight of the one or more acrylate thickeners.

31. The preparation of claim 28, wherein the preparation comprises a total of from 1.5% to 3% by weight of the one or more acrylate thickeners.

32. The preparation of claim 15, wherein the one or more acrylate thickeners comprise one or more copolymers comprising units of (i) one or more acrylate monomers selected from acrylic acid, methacrylic acid, itaconic acid, fumaric acid, crotonic acid, aconitic acid and maleic acid, (ii) one or more α,β-ethylenically unsaturated monomers of formula CH₂═CXY wherein X═H, CH₃, C₁-C₃₀-alkyl, —CH₂—C(═O)O(CH₂—CH₂—O)ₓ—R³, —CH₂—C(═O)NH(CH₂—CH₂—O)ₓ—R³, —CH₂—CH₂—(CH₂—CH₂—O)ₓ—R³ with x=1-100 and R³=C₁-C₃₀-alkyl or Cl und Y=—COOR, —C₆H₄R, —CN, —CONH₂, —Cl, —NC₄H₆O, —NH(CH₂)₃COOH, —NHCOCH₃, —CONHC(CH₃)₃, —CON(CH₃)₂, —CH═CH₂, C₁-C₁₈-alkyl, hydroxy-C₁-C₁₈-alkyl, —C(═O)O(CH₂—CH₂—O)ₓ—R³, —C(═O)NH(CH₂—CH₂—O)ₓ—R³, —CH₂═(CH₂—CH₂—O)ₓ—R³ with x=1-100 and R³=C₁-C₃₀-alkyl or of formula CH₂═CH(OCOR²) with R²=C₁-C₁₈-alkyl or of formula CH₂═CH₂ or CH₂═CHCH₃ and (iii) one or more polyunsaturated monomers which are suitable for partial crosslinking.

33. The preparation of claim 17, wherein the preparation comprises from 0.01% to 3% by weight of polyvinylpyrrolidone, based on a total weight of the preparation.

34. The preparation of claim 33, wherein the preparation comprises from 0.1% to 1% by weight of polyvinylpyrrolidone.

35. The preparation of claim 33, wherein the preparation comprises from 0.1% to 0.9% by weight of polyvinylpyrrolidone.

36. The preparation of claim 20, wherein the preparation comprises a total of from 0.01% to 0.6% by weight of the one or more cationic surfactants, based on a total weight of the preparation.

37. The preparation of claim 36, wherein the preparation comprises a total of from 0.1% to 0.3% by weight of the one or more cationic surfactants.

38. The preparation of claim 20, wherein the one or more cationic surfactants are selected from one or more of polyquatemium-7, polyquatemium-10, polyquatemium-44 and guar quats or are selected from a combination of one or more polyquats and one or more guar quats.

39. The preparation of claim 20, wherein the one or more cationic surfactants comprise at least one of polyquatemium-10 and guar quat and wherein the preparation is adapted for cleansing hair and scalp.

40. The preparation of claim 39, wherein the only cationic surfactant present in the preparation is polyquaternium-10.

41. The preparation of claim 15, wherein the preparation further comprises one or more nonionic surfactants having an HLB value of from 10 to 20.

42. A cosmetic or dermatological washing-active preparation for cleansing skin and/or hair and scalp, wherein the preparation comprises, based on a total weight of the preparation:
(a) from 5% to 15% by weight of one or more anionic surfactants;
(b) from 0.5% to 10% by weight of one or more amphoteric surfactants;
(c) from 0.1% to 8.0% by weight of one or more gel-forming acrylate thickeners selected from crosslinked alkali-swellable acrylate copolymers; and
(d) from 0.01% to 3% by weight of polyvinylpyrrolidone.

43. The preparation of claim 42, wherein (a) comprises at least one of lauryl ether sulfate and myreth ether sulfate and (b) comprises cocoamidopropylbetaine.

44. The preparation of claim 42, wherein the preparation further comprises from 0.01% to 0.6% by weight of one or more cationic surfactants, based on a total weight of the preparation.

45. The preparation of claim 42, wherein the preparation further comprises one or more nonionic surfactants having an HLB value of from 10 to 20.

46. The preparation of claim 42, wherein the preparation comprises a total of up to 12% by weight of surfactants, based on a total weight of the preparation.

47. The preparation of claim 44, wherein the one or more cationic surfactants are selected from one or more of polyquatemium-7, polyquatemium-10, polyquatemium-44 and guar quats or are selected from a combination of one or more polyquats and one or more guar quats.

48. A cosmetic or dermatological washing-active preparation for cleansing skin and/or hair and scalp, wherein the preparation comprises, based on a total weight of the preparation:
(a) from 6% to 12% by weight of one or more anionic surfactants which comprise at least one of lauryl ether sulfate and myreth ether sulfate;
(b) from 1% to 5% by weight of one or more amphoteric surfactants which comprise cocoamidopropylbetaine;
(c) from 0.5% to 4% by weight of one or more gel-forming acrylate thickeners selected from crosslinked alkali-swellable acrylate copolymers; and
(d) from 0.1% to 1% by weight of polyvinylpyrrolidone.

49. The preparation of claim 48, wherein the preparation further comprises from 0.1% to 0.3% by weight of one or more cationic surfactants, based on a total weight of the preparation, the one or more cationic surfactants being selected from one or more of polyquatemium-7, polyquatemium-10, polyquatemium-44 and guar quats or being selected from a combination of one or more polyquats and one or more guar quats.
